(19) United States
(12) Patent Application Publication
(10) Pub. No.: US 2023/0129327 A1
(43) Pub. Date: Apr. 27, 2023

(54) APPARATUS FOR PROVIDING CUSTOMIZED NUTRITIONAL SUPPLEMENT INFORMATION AND OPERATION METHOD THEREOF

(71) Applicant: Aimee Labs Co., Ltd., Seoul (KR)

(72) Inventors: Min Suk KIM, Seoul (KR); Hye Yong KIM, Seoul (KR)

(73) Assignee: Aimee Labs Co., Ltd., Seoul (KR)

(21) Appl. No.: 17/970,099

(22) Filed: Oct. 20, 2022

(30) Foreign Application Priority Data
Oct. 21, 2021 (KR) 10-2021-0141172

(51) Int. Cl. G16H 20/60; G16H 10/60; G16H 10/20; G09B 19/00

(52) U.S. Cl. CPC G16H 20/60; G09B 19/0092; G16H 10/20; G16H 10/60

(57) ABSTRACT

Disclosed is a technology of providing customized nutritional supplement information. More particularly, provided is a device for providing nutritional supplement information, including: a nutritional supplement database configured to store intaking nutritional information according to a person’s age and gender, and information on a commercially available nutritional supplement; a member manager configured to generate member information by registering a user of a nutritional supplement information-providing service and a counselor member for professional consultation and, when a service use request is received from a user terminal, and to perform member authentication for whether the user is registered as a member based on user’s identification information included in the service use request; a nutritional supplement recommender configured to generate questionnaire data for recommending a nutritional supplement to the user when the user is confirmed as a registered member, and to generate user-customized nutritional supplement recommendation information based on answer data to the questionnaire data received from the user terminal and the intaking nutritional information; and a communicator configured to transmit the nutritional supplement recommendation information to the user terminal.

### CROSS-REFERENCE TO RELATED APPLICATION

This application claims priority to Korean Patent Application No. 10-2021-0141172, filed on Oct. 21, 2021 in the Korean Intellectual Property Office, the disclosure of which is incorporated herein by reference.

### BACKGROUND OF THE DISCLOSURE

### Field of the Disclosure

The present disclosure relates to a technology for providing customized nutritional supplement information.

### Description of the Related Art

Recently, the demand and supply of nutritional supplements for supplementing essential nutrients of humans are increasing as interest in health problems has rapidly increased. Since the amount of nutrients that a person must consume is set at an appropriate level, indiscriminate intake of nutritional supplements can adversely affect the body.

In addition, since the amount of nutrients required for a person is different according to a health condition, diet, age, sex, etc., there is a need to provide information on intake of appropriate nutrients to each person.

### RELATED ART DOCUMENT

### Patent Document

(Patent Document 1) Korean Patent Application Publication No. 10-2021-0110006 “SERVER AND METHOD FOR RECOMMENDING PRODUCT”

### SUMMARY OF THE DISCLOSURE

Therefore, the present disclosure has been made in view of the above problems, and it is an object of the present disclosure to provide a customized nutritional supplement recommendation service for each person.

In accordance with an aspect of the present disclosure, the above and other objects can be accomplished by the provision of a device for providing nutritional supplement information, including: a nutritional supplement database configured to store intaking nutritional information according to a person’s age and gender, and information on a commercially available nutritional supplement; a member manager configured to generate member information by registering a user of a nutritional supplement information-providing service and a counselor member for professional consultation and, when a service use request is received from a user terminal, and to perform member authentication for whether the user is registered as a member based on user’s identification information included in the service use request; a nutritional supplement recommender configured to generate questionnaire data for recommending a nutritional supplement to the user when the user is confirmed as a registered member, and to generate user-customized nutritional supplement recommendation information based on answer data to the questionnaire data received from the user terminal and the intaking nutritional information; and a communicator configured to transmit the nutritional supplement recommendation information to the user terminal.

In one embodiment, the device for providing nutritional supplement information may further include a nutritional supplement comparator configured to generate nutritional supplement comparison information for comparing ingredients of nutritional supplements including a nutrient necessary for the user based on the nutritional supplement recommendation information and the commercially available nutritional supplement information when a nutritional supplement comparison information request is received from a user terminal that has received the nutritional supplement recommendation information, wherein the communicator transmits the nutritional supplement comparison information to the user terminal.

In one embodiment, the device for providing nutritional supplement information may further include a consultation manager configured to identify a counselor available for consultation among registered counselor members when a consultation request is received from the user terminal that has received the nutritional supplement comparison information, wherein the communicator transmits information on the counselor confirmed to be available for consultation to the user terminal.

In one embodiment, the nutritional supplement database may update member information of the user based on age and gender information of the user, the answer data, the nutritional supplement recommendation information, the nutritional supplement comparison information, and the information on the counselor confirmed to be available for consultation.

In one embodiment, the communicator may request intake state information of the user according to the nutritional supplement recommendation information to the user terminal and receive the intake state information of the user from the user terminal, and the nutritional supplement database may update the member information of the user based on the intake state information of the user.

In one embodiment, the intaking nutritional information may include person’s nutritional supplement intaking information including a person’s age, gender, height, weight, pregnancy, lactation, underlying medical condition, alcohol consumption, smoking status, outdoor activity type and duration, sleep duration, diet, taking nutritional supplement, and information on taking medicine and purpose thereof.

In one embodiment, the nutritional supplement information may include a product name, main ingredient, supplementary ingredient, dosage form, a content by raw material, manufacturer, raw material company, brand, product image, selling price, vendor information, and medication guide of the commercially available nutritional supplement.

In one embodiment, the answer data may include an age, gender, height, weight, pregnancy, lactation, underlying medical condition, alcohol consumption, smoking status, outdoor activity type and duration, sleep duration, diet, taking nutritional supplement, information on a taking medicine and a purpose thereof of the user.

In one embodiment, nutritional supplement recommendation information may include a product name, main ingredient, supplementary ingredient, dosage form, a content by raw material, manufacturer, raw material company, brand, product image, selling price, vendor information, and medication guide of a nutritional supplement necessary for the user.

In one embodiment, the answer data may include personal preference information including at least one of a dosage form, size, odor, medication guide, vegan certification information, gluten-free certification information, and GMO-free certification information of a nutritional supplement.

In one embodiment, the nutritional supplement comparison information request may further include product name information of nutritional supplements that the user wants to compare, wherein the nutritional supplement comparison information includes ingredient contents, dosage forms, medication guides, and efficacy comparison information of nutritional supplements corresponding to the product name information.

In accordance with another aspect of the present disclosure, there is provided a method performed using a device for providing nutritional supplement information, the method including: generating a nutritional supplement database for storing intaking nutritional information according to a person’s age and gender, and information on a commercially available nutritional supplement; generating a member manager for generating member information by registering a user of a nutritional supplement information-providing service and a counselor member for professional consultation; receiving the nutritional supplement information-providing service use request from a user terminal; performing member authentication for whether the user is registered as a member based on user’s identification information included in the service use request; transmitting questionnaire data for recommending a nutritional supplement to the user terminal when the user is confirmed as a registered member; receiving answer data to the questionnaire data from the user terminal; generating user-customized nutritional supplement recommendation information based on the answer data and the intaking nutritional information; and transmitting the nutritional supplement recommendation information to the user terminal.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a view for explaining a system for providing nutritional supplement information according to an embodiment of the present disclosure;

FIG. 2 illustrates a flowchart of a method of providing nutritional supplement information according to an embodiment of the present disclosure;

FIG. 3 illustrates a block diagram of a device for providing nutritional supplement information according to an embodiment of the present disclosure;

FIG. 4 is a view for explaining a system for providing nutritional supplement information according to another embodiment of the present disclosure; and

FIG. 5 illustrates a block diagram of a user terminal, a device for providing nutritional supplement information, and a counselor terminal according to another embodiment of the present disclosure.

### DETAILED DESCRIPTION OF THE DISCLOSURE

The embodiments will be described in detail herein with reference to the drawings.

However, it should be understood that the present disclosure is not limited to the embodiments according to the concept of the present disclosure, but includes changes, equivalents, or alternatives falling within the spirit and scope of the present disclosure.

In the following description of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. Accordingly, definitions of the terms should be understood on the basis of the entire description of the present specification.

In description of the drawings, like reference numerals may be used for similar elements.

The singular expressions in the present specification may encompass plural expressions unless clearly specified otherwise in context.

In this specification, expressions such as “A or B” and “at least one of A and/or B” may include all possible combinations of the items listed together.

Expressions “first” and “second” can be used to qualify the components, regardless of order or importance, and are used to distinguish one component from another, and do not limit the components.

It should be understood that when an element (e.g., first) is referred to as being “connected to” or “coupled to” another element (e.g., second), the element may be directly connected or coupled to the other element or intervening element (e.g., a third element) may be present.

As used herein, “configured to” may be used interchangeably with, for example, “suitable for”, “ability to”, “changed to”, “made to”, “capable of”, or “designed to” in terms of hardware or software.

In some situations, the expression “device configured to” may mean that the device “may do ∼” with other devices or components.

For example, the expression “processor configured to perform A, B, and C” may mean that a general-purpose processor (e.g., CPU or application processor) performs the corresponding operations by executing a dedicated processor (e.g., an embedded processor) or one or more software programs stored in the memory device.

In addition, the expression “or” means “inclusive or” rather than “exclusive or”.

That is, unless otherwise mentioned or clearly inferred from context, the expression “x uses a or b” means any one of natural inclusive permutations.

Terms, such as “unit” or “module”, etc., should be understood as a unit that processes at least one function or operation and that may be embodied in a hardware manner, a software manner, or a combination of the hardware manner and the software manner.

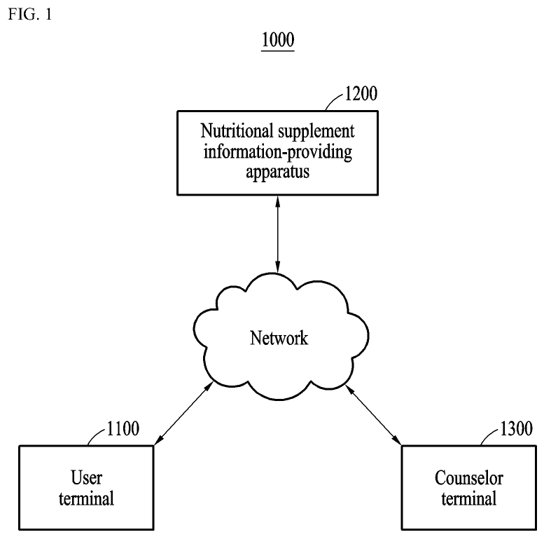

FIG. 1 is a view for explaining a system for providing nutritional supplement information according to an embodiment of the present disclosure.

Referring to FIG. 1, a system 1000 for providing nutritional supplement information may include a user terminal 1100, a nutritional supplement information-providing apparatus 1200, and a counselor terminal 1300. Here, the user terminal 1100, the nutritional supplement information-providing apparatus 1200, and the counselor terminal 1300 may be connected to each other to enable data exchange with each other through a wired/wireless communication network.

The user terminal 1100 may mean a terminal used by a user who wants to use a nutritional supplement information providing service. The user terminal 1100 may receive information for using a nutritional supplement information-providing service from a user, and may transmit the inputted information to the nutritional supplement information-providing apparatus 1200. The user terminal 1100 may output the information received from the nutritional supplement information-providing apparatus 1200 on a display such that the user can recognize the information.

The nutritional supplement information-providing apparatus 1200 may provide information related to nutritional supplements. The nutritional supplement information-providing apparatus 1200 may receive the user information from the user terminal 1100. the nutritional supplement information-providing apparatus 1200 may generate user-customized nutritional supplement recommendation information based on the information received from the user terminal 1100. The nutritional supplement information-providing apparatus 1200 may transmit the generated nutritional supplement recommendation information to the user terminal 1100.

The counselor terminal 1300 may refer to a terminal used by experts (hereinafter referred to as “counselor”) such as doctors and pharmacists. The counselor terminal 1300 may enable a nutritional supplement-related consultation between the user and the counselor by enabling calls, chatting, etc. with the user terminal 1100 when there is a user’s request.

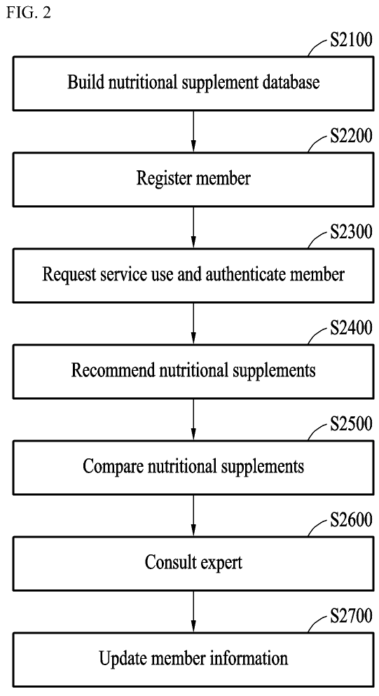

Hereinafter, the system for providing nutritional supplement information is described in more detail with reference to FIGS. 2 to 5.

FIG. 2 illustrates a flowchart of a method of providing nutritional supplement information according to an embodiment of the present disclosure.

Hereinafter, the method illustrated in FIG. 2 is described as an example of a method performed by the system 1000 for providing nutritional supplement information illustrated in FIG. 1.

In S2100, a nutritional supplement database is built. Specifically, the nutritional supplement information-providing apparatus 1200 may store intaking nutritional information recommended according to the age, gender, etc. of a person in a nutritional supplement database.

In one embodiment, the intaking nutritional information may differ depending upon a person’s age, gender, race, height, weight, pregnancy, lactation, underlying medical condition, alcohol consumption, smoking status, outdoor activity type and duration, sleep duration, diet, taking nutritional supplement, and information on taking medicine and purpose (e.g., recovery from fatigue, liver health, intestinal health) thereof, the presence or absence of allergies, and the like.

In addition, the nutritional supplement information-providing apparatus 1200 may store information on commercially available nutritional supplements in the nutritional supplement database.

In one embodiment, the nutritional supplement information may include information such as nutritional supplement’s product name, main ingredient, supplementary ingredient, dosage form, size, odor, taking method, efficacy, content by raw material, manufacturer, raw material company, brand, product image, selling price, vendor information, side effects, and allergy-causing possibility. Here, the content by raw material may include information on the presence or absence of specific raw materials or ingredients such as vegan certification, gluten-free certification, and genetically modified food (GMO)-free certification.

In S2200, a member using the nutritional supplement information-providing service is registered. Specifically, identification information such as the name, age, phone number, account number, and credit card number of the user may be inputted in the user terminal 1100 by the user. The user terminal 1100 may transmit a nutritional supplement information-providing service subscription application including the inputted user’s identification information to the nutritional supplement information-providing apparatus 1200. The nutritional supplement information-providing apparatus 1200 may receive the nutritional supplement information-providing service subscription application from the user terminal 1100. The nutritional supplement information-providing apparatus 1200 may generate member information based on the user’s identification information included in the nutritional supplement information-providing service subscription application to register the user as a user member. When the member registration is completed, the nutritional supplement information-providing apparatus 1200 may transmit a confirmation message indicating that the member registration is completed to the user terminal 1100.

In addition, the nutritional supplement information-providing apparatus 1200 may register specialists, such as doctors and pharmacists who can provide professional consultation on nutritional supplements, as counselor members. Specifically, the counselor may input identification information such as name, resident number, phone number, account number, and credit card number, and a doctor’s license, a pharmacist’s license, and other related degree information to the counselor terminal 1300. The counselor terminal 1300 may transmit the counselor registration request including the information inputted from counselor to the nutritional supplement information-providing apparatus 1200. The nutritional supplement information-providing apparatus 1200 may receive the counselor registration request from the counselor terminal 1300. The nutritional supplement information-providing apparatus 1200 may register the counselor member by generating member information based on information included in the counselor registration request. When the member registration is completed, the nutritional supplement information-providing apparatus 1200 may transmit a confirmation message indicating that the member registration of the counselor is completed to the counselor terminal 1300.

In S2300, a service use request and member authentication are performed. Specifically, the user terminal 1100 may transmit the service use request including the user’s identification information to the nutritional supplement information-providing apparatus 1200. The nutritional supplement information-providing apparatus 1200 may receive the service use request from the user terminal 1100. The nutritional supplement information-providing apparatus 1200 may perform member authentication to check whether a member is registered in the nutritional supplement information-providing service based on the user’s identification information included in the service use request.

In one embodiment, when it is confirmed that the member is not registered, the nutritional supplement information-providing apparatus 1200 may transmit a message indicating that member registration is required to the user terminal 1100.

In S2400, a nutritional supplement necessary for the user is recommended. Specifically, the nutritional supplement information-providing apparatus 1200 may transmit questionnaire data for nutritional supplement recommendation to the user terminal 1100 when the user is confirmed as a registered member. The user terminal 1100 may receive the questionnaire data from the nutritional supplement information-providing apparatus 1200. The user terminal 1100 may display the received questionnaire data on a display such that the user can recognize the received questionnaire data. The user may input an answer corresponding to the questionnaire data to the user terminal 1100 to generate answer data. The user terminal 1100 may transmit the answer data to the nutritional supplement information-providing apparatus 1200. The nutritional supplement information-providing apparatus 1200 may receive the answer data from the user terminal 1100. The nutritional supplement information-providing apparatus 1200 may generate nutritional supplement recommendation information, which includes nutritional supplement information including a nutrient necessary for the user, based on the intaking nutritional information, nutritional supplement information, and the like stored in the answer data and the database. The nutritional supplement information-providing apparatus 1200 may transmit the nutritional supplement recommendation information to the user terminal 1100. The user terminal 1100 may receive the nutritional supplement recommendation information from the nutritional supplement information-providing apparatus 1200, and may output the nutritional supplement recommendation information on a display such that the user can recognize.

In one embodiment, the questionnaire data may be question data asking for a user’s age, gender, height, weight, pregnancy, lactation, underlying disease, drinking, smoking, outdoor activity type and time, sleeping time, eating habit, nutritional supplement information, and medication information, and the answer data may be data including an answer to the questionnaire data. In addition, the question data may further include questions asking for personal preference information such as nutritional supplement attribute information, raw material attribute information. Here, the nutritional supplement attribute information may refer to externally revealed characteristics of a nutritional supplement such as a dosage form, the size, odor, and taking method of a pill, and an administration method thereof. The raw material attribute information may refer to information on the presence or absence of specific raw materials or ingredients such as vegan certification, gluten-free certification, and genetically modified food (GMO)-free certification.

In one embodiment, the nutritional supplement recommendation information may include information such as the product name, main ingredient, supplementary ingredient, dosage form, size, odor, taking method, efficacy, content by raw material, manufacturer, raw material company, brand, product image, selling price, vendor information, side effects, and allergy-causing possibility of a nutritional supplement necessary for the user. Here, the content by raw material may include information on the presence or absence of specific raw materials or ingredients such as vegan certification, gluten-free certification, and genetically modified food (GMO)-free certification.

In S2500, the nutritional supplement comparison information is provided. Specifically, product name information of a nutritional supplement to be compared may be inputted to the user terminal 1100 by the user. The user terminal 1100 may transmit a nutritional supplement comparison request including the product name information inputted by the user to the nutritional supplement information-providing apparatus 1200. The nutritional supplement information-providing apparatus 1200 may generate nutritional supplement comparison information based on the nutritional supplement information stored in the database. Here, nutritional supplements to be compared may be nutritional supplements corresponding to the product name information included in the nutritional supplement comparison request. The nutritional supplement information-providing apparatus 1200 may transmit the nutritional supplement comparison information to the user terminal 1100. The user terminal 1100 may receive the nutritional supplement comparison information from the nutritional supplement information-providing apparatus 1200, and may output the received nutritional supplement comparison information on a display such that the user can recognize.

In one embodiment, the product name information inputted by the user may be at least one of nutritional supplements included in the nutritional supplement recommendation information.

In one embodiment, the nutritional supplement comparison information may include ingredient and price comparison information on nutritional supplements.

In S2600, expert consultation is performed. Specifically, a command of requesting expert consultation may be inputted to the user terminal 1100 by the user. The user terminal 1100 may transmit the expert consultation request to the nutritional supplement information-providing apparatus 1200. The nutritional supplement information-providing apparatus 1200 may receive the expert consultation request from the user terminal 1100. The nutritional supplement information-providing apparatus 1200 may transmit a consultation availability confirmation request of experts registered as counselor members to the counselor terminal 1300. The counselor terminal 1300 may receive the consultation availability confirmation request from the nutritional supplement information-providing apparatus 1200, and may output the received consultation availability confirmation request on a display such that the counselor can recognize. A confirmation of whether consultation is possible is inputted to the counselor terminal 1300 by the counselor, and may transmit the inputted confirmation to the nutritional supplement information-providing apparatus 1200. The nutritional supplement information-providing apparatus 1200 may receive the confirmation of whether consultation is possible from the counselor terminal 1300. The nutritional supplement information-providing apparatus 1200 may transmit information of the counselor available for consultation to the user terminal 1100. The user terminal 1100 may receive the counselor information from the nutritional supplement information-providing apparatus 1200, and may display the received counselor information on a display such that the user can recognize.

In one embodiment, the expert consultation request may further include a purpose (e.g., allergy, underlying disease, etc.) for which the user wants to consult. Here, the nutritional supplement information-providing apparatus 1200 may check whether a counselor with specialized knowledge corresponding to the purpose for which the user wants to consult is available.

In one embodiment, the counselor information may include a phone number, a messenger ID, a chatting consultation connection link, and the like.

In S2700, the member information is updated. Specifically, the nutritional supplement information-providing apparatus 1200 may add the nutritional supplement recommendation information, nutritional supplement comparison information, counselor information, and the like transmitted to the user terminal 1100 to the member information of the user to update member information.

In one embodiment, whether the intake of the nutritional supplement according to the nutritional supplement recommendation information may be inputted to the user terminal 1100 by the user. The user terminal 1100 may generate intake state information based on whether the intake of the nutritional supplement inputted from the user, and may transmit the generated intake state information to the nutritional supplement information-providing apparatus 1200. The nutritional supplement information-providing apparatus 1200 may receive the intake state information from the user terminal 1100, and may update the member information based on the received intake state information.

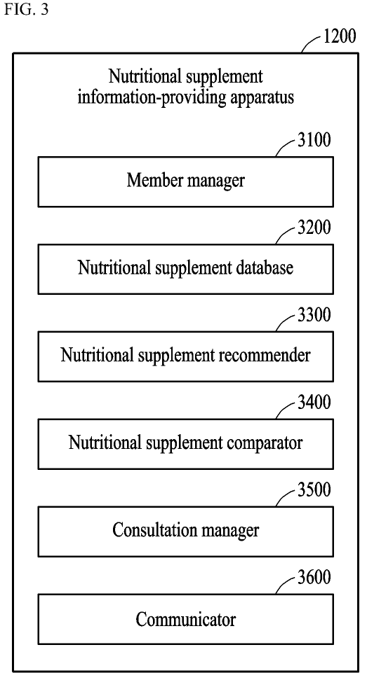

FIG. 3 illustrates a block diagram of a device for providing nutritional supplement information according to an embodiment of the present disclosure.

Referring to FIG. 3, the nutritional supplement information-providing apparatus 1200 may include a member manager 3100, a nutritional supplement database 3200, a nutritional supplement recommender 3300, a nutritional supplement comparator 3400, a consultation manager 3500, and a communicator 3600.

The member manager 3100 may register a user of the nutritional supplement information-providing service and a counselor member for professional consultation to generate member information. When a service use request received from the user terminal 1100 is received, the member manager 3100 may perform member authentication for whether the user is registered as a member based on user’s identification information included in the service use request.

The nutritional supplement recommender 3300 may generate questionnaire data for recommending a nutritional supplement to the user. The nutritional supplement recommender 3300 may generate answer data to the questionnaire data received from the user terminal 1100; and user-customized nutritional supplement recommendation information based on the intaking nutritional information.

The nutritional supplement database 3200 may store intaking nutritional information according to a person’s age and gender; and information on commercially available nutritional supplements.

In one embodiment, the nutritional supplement database 3200 may add the nutritional supplement recommendation information, the nutritional supplement comparison information, the counselor information, and the like to the member information of the user to update the member information.

In one embodiment, the nutritional supplement database 3200 may update the member information based on intake state information.

The nutritional supplement comparator 3400 may generate nutritional supplement comparison information that compares ingredients of nutritional supplements including a nutrient necessary for the user, based on the nutritional supplement recommendation information and the information on commercially available nutritional supplements.

When a consultation request is received from the user terminal 1100, the consultation manager 3500 may identify counselors available for consultation among the registered counselor members.

The communicator 3600 may perform data communication between the nutritional supplement information-providing apparatus 1200 and the user terminal 1100 and the counselor terminal 1300.

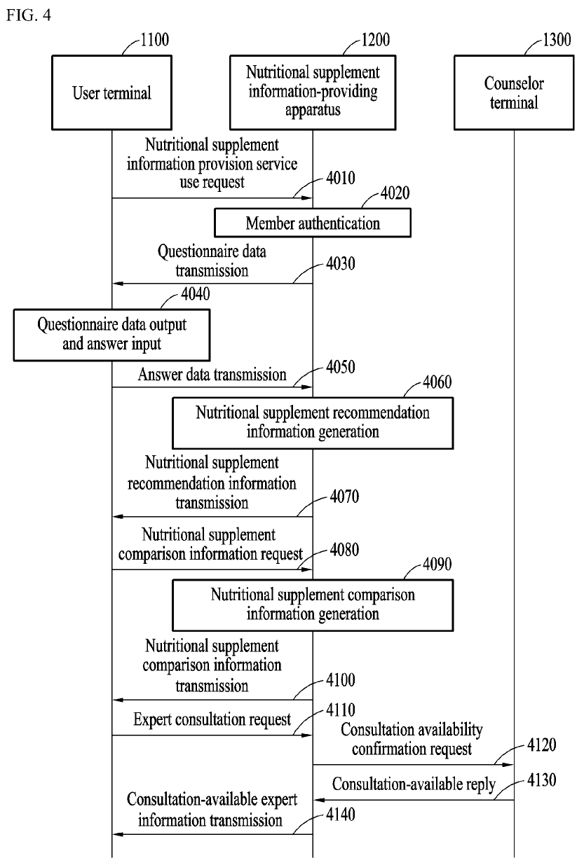

FIG. 4 is a view for explaining a system for providing nutritional supplement information according to another embodiment of the present disclosure.

In S4010, the user terminal 1100 may transmit a nutritional supplement information-providing service use request to the nutritional supplement information-providing apparatus 1200.

In S4020, the nutritional supplement information-providing apparatus 1200 may perform member authentication for the nutritional supplement information-providing service use request when the nutritional supplement information-providing service use request is received form the user terminal 1100.

In S4030, when the member authentication is completed, the nutritional supplement information-providing apparatus 1200 may transmit the questionnaire data for nutritional supplement recommendation to the user terminal 1100.

In S4040, when the questionnaire data is received from the nutritional supplement information-providing apparatus 1200, the user terminal 1100 may output the questionnaire data on a display such that the user can recognize. An answer corresponding to the questionnaire data may be inputted to the user terminal 1100 by the user, and answer data may be generated based on the inputted answer.

In S4050, the user terminal 1100 may transmit the answer data to the nutritional supplement information-providing apparatus 1200.

In S4060, when the answer data is received from the user terminal 1100, the nutritional supplement information-providing apparatus 1200 may generate nutritional supplement recommendation information including information on a nutritional supplement that the user should consume, based on the answer data.

In S4070, the nutritional supplement information-providing apparatus 1200 may transmit the nutritional supplement recommendation information to the user terminal 1100. Here, the user terminal 1100 may output the nutritional supplement recommendation information, received from the nutritional supplement information-providing apparatus 1200, on a display such that the user can recognize.

In S4080, when a command for requesting comparison information on nutritional supplements is inputted by the user, the user terminal 1100 may transmit the nutritional supplement comparison information request to the nutritional supplement information-providing apparatus 1200.

In S4090, when the nutritional supplement comparison information request is received from the user terminal 1100, the nutritional supplement information-providing apparatus 1200 may generate nutritional supplement comparison information, which compares the ingredients, prices, and the like of nutritional supplements, comparison-requested from the user terminal 1100 based on the nutritional supplement information stored in the nutritional supplement database.

In S4100, the nutritional supplement information-providing apparatus 1200 may transmit the nutritional supplement comparison information to the user terminal 1100. Here, when the nutritional supplement comparison information is received from the nutritional supplement information-providing apparatus 1200, the user terminal 1100 may output the received nutritional supplement comparison information on a display such that the user can recognize the received nutritional supplement comparison information.

In S4110, when a command that expert consultation is required is inputted from the user, the user terminal 1100 may transmit the expert consultation request to the nutritional supplement information-providing apparatus 1200.

In S4120, when the expert consultation request is received from the user terminal 1100, the nutritional supplement information-providing apparatus 1200 may transmit the consultation availability confirmation request of the counselors registered as members to the counselor terminal 1300.

In S4130, the counselor terminal 1300 may output the consultation availability confirmation request on a display such that the counselor can recognize the consultation availability confirmation request when the consultation availability confirmation request is received from the nutritional supplement information-providing apparatus 1200. When a command to confirm whether consultation is possible is inputted from the counselor, the counselor terminal 1300 may transmit the reply on whether consultation is possible to the nutritional supplement information-providing apparatus 1200.

In S4140, when the consultation-available reply is received from the counselor terminal 1300, the nutritional supplement information-providing apparatus 1200 may transmit information of the counselor available for consultation to the user terminal 1100. Here, the user terminal 1100 may output the counselor information received from the nutritional supplement information-providing apparatus 1200 on a display such that the user can recognize the counselor information.

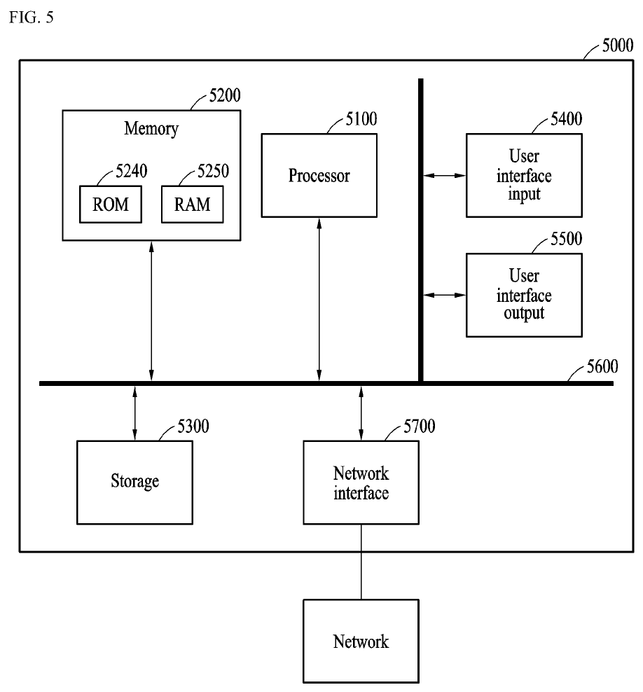

FIG. 5 illustrates a block diagram of a user terminal, a device for providing nutritional supplement information, and a counselor terminal according to another embodiment of the present disclosure.

As shown in FIG. 5, the user terminal 1100, the nutritional supplement information-providing apparatus 1200, and the counselor terminal 1300 may include at least one element of a processor 5100, a memory 5200, a storage 5300, a user interface input 5400, and a user interface output 5500, and these elements may communicate each other via a bus 5600. In addition, the user terminal 1100, the nutritional supplement information-providing apparatus 1200, and the counselor terminal 1300 may also include a network interface 5700 for accessing a network. The processor 5100 may be a CPU or semiconductor device that executes processing instructions stored in the memory 5200 and/or the storage 5300. The memory 5200 and the storage 5300 may include various types of volatile/nonvolatile memory media. For example, the memory may include ROM 5240 and RAM 5250.

According to an embodiment of the present disclosure, a customized nutritional supplement recommendation service for each individual can be provided.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable array (FPA), a programmable logic unit (PLU), a microprocessor, or any other device capable of executing and responding to instructions. The processing device may execute an operating system (OS) and one or more software applications executing on the operating system.

In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

### DESCRIPTION OF SYMBOLS

1000: a system for providing nutritional supplement information 1100: a user terminal 1200: a device for providing nutritional supplement information 1300: a counselor terminal

What is claimed is:

1. A device for providing nutritional supplement information, comprising:
a nutritional supplement database configured to store intaking nutritional information according to a person’s age and gender, and information on a commercially available nutritional supplement;
a member manager configured to generate member information by registering a user of a nutritional supplement information-providing service and a counselor member for professional consultation and, when a service use request is received from a user terminal, and to perform member authentication for whether the user is registered as a member based on user’s identification information comprised in the service use request;
a nutritional supplement recommender configured to generate questionnaire data for recommending a nutritional supplement to the user when the user is confirmed as a registered member, and to generate user-customized nutritional supplement recommendation information based on answer data to the questionnaire data received from the user terminal and the intaking nutritional information; and
a communicator configured to transmit the nutritional supplement recommendation information to the user terminal.

2. The device according to claim 1, further comprising:
when a nutritional supplement comparison information request comprising product name information of nutritional supplements to be compared is received from the user terminal, a nutritional supplement comparator configured to generate nutritional supplement comparison information for comparing ingredients of nutritional supplements corresponding to product name information of the nutritional supplements based on the commercially available nutritional supplement information,
wherein the communicator transmits the nutritional supplement comparison information to the user terminal.

3. The device according to claim 2, further comprising:
when a consultation request is received from the user terminal that has received the nutritional supplement comparison information, a consultation manager configured to identify a counselor available for consultation among registered counselor members,
wherein the communicator transmits information on the counselor confirmed to be available for consultation to the user terminal.

4. The device according to claim 3, wherein the nutritional supplement database updates member information of the user based on age and gender information of the user, the answer data, the nutritional supplement recommendation information, the nutritional supplement comparison information, and the information on the counselor confirmed to be available for consultation.

5. The device according to claim 4, wherein the communicator requests intake state information of the user according to the nutritional supplement recommendation information to the user terminal and receives the intake state information of the user from the user terminal, and
the nutritional supplement database updates the member information of the user based on the intake state information of the user.

6. The device according to claim 1, wherein the intaking nutritional information comprises person’s nutritional supplement intaking information comprising a person’s age, gender, height, weight, pregnancy, lactation, underlying medical condition, alcohol consumption, smoking status, outdoor activity type and duration, sleep duration, diet, taking nutritional supplement, and information on taking medicine and purpose thereof,
the nutritional supplement information comprises a product name, main ingredient, supplementary ingredient, dosage form, a content by raw material, manufacturer, raw material company, brand, product image, selling price, vendor information, and medication guide of the commercially available nutritional supplement,
the answer data comprises an age, gender, height, weight, pregnancy, lactation, underlying medical condition, alcohol consumption, smoking status, outdoor activity type and duration, sleep duration, diet, taking nutritional supplement, information on a taking medicine and a purpose thereof of the user, and
the nutritional supplement recommendation information comprises a product name, main ingredient, supplementary ingredient, dosage form, a content by raw material, manufacturer, raw material company, brand, product image, selling price, vendor information, and medication guide of a nutritional supplement necessary for the user.

7. The device according to claim 1, wherein the answer data comprises personal preference information comprising at least one of a dosage form, size, odor, medication guide, vegan certification information, gluten-free certification information, and GMO-free certification information of a nutritional supplement.

8. The device according to claim 2, wherein the nutritional supplement comparison information request further comprises product name information of nutritional supplements that the user wants to compare,
wherein the nutritional supplement comparison information comprises ingredient contents, dosage forms, medication guides, and efficacy comparison information of nutritional supplements corresponding to the product name information.

9. A method performed using a device for providing nutritional supplement information, the method comprising:
generating a nutritional supplement database for storing intaking nutritional information according to a person’s age and gender, and information on a commercially available nutritional supplement;
generating a member manager for generating member information by registering a user of a nutritional supplement information-providing service and a counselor member for professional consultation;
receiving the nutritional supplement information-providing service use request from a user terminal;
performing member authentication for whether the user is registered as a member based on user’s identification information comprised in the service use request;
transmitting questionnaire data for recommending a nutritional supplement to the user terminal when the user is confirmed as a registered member;
receiving answer data to the questionnaire data from the user terminal;
generating user-customized nutritional supplement recommendation information based on the answer data and the intaking nutritional information; and
transmitting the nutritional supplement recommendation information to the user terminal.

10. The method according to claim 9, further comprise:
receiving a comparison information request comprising product name information of nutritional supplements to be compared from the user terminal;
generating nutritional supplement comparison information to compare ingredient contents, dosage forms, medication guides, and efficacy of nutritional supplements corresponding to the product name information based on the commercially available nutritional supplement information; and
transmitting the nutritional supplement comparison information to the user terminal.

11. The method according to claim 10, further comprising:
receiving a consultation request from the user terminal that has received the nutritional supplement comparison information;
identifying a counselor available for consultation among registered counselor members; and
transmitting information on the counselor confirmed to be available for consultation to the user terminal.

12. The method according to claim 11, further comprising updating member information of the user based on age and gender information of the user, the answer data, the nutritional supplement recommendation information, the nutritional supplement comparison information, and the information on the counselor confirmed to be available for consultation.

13. The method according to claim 12, further comprising:
requesting intake state information of the user according to the nutritional supplement recommendation information to the user terminal;
receiving the intake state information of the user from the user terminal; and
updating the member information of the user based on the intake state information of the user.

14. The method according to claim 9, wherein the intaking nutritional information comprises person’s nutritional supplement intaking information comprising a person’s age, gender, height, weight, pregnancy, lactation, underlying medical condition, alcohol consumption, smoking status, outdoor activity type and duration, sleep duration, diet, taking nutritional supplement, and information on taking medicine and purpose thereof,
the nutritional supplement information comprises a product name, main ingredient, supplementary ingredient, dosage form, a content by raw material, manufacturer, raw material company, brand, product image, selling price, vendor information, and medication guide of the commercially available nutritional supplement,
the answer data comprises an age, gender, height, weight, pregnancy, lactation, underlying medical condition, alcohol consumption, smoking status, outdoor activity type and duration, sleep duration, diet, taking nutritional supplement, information on a taking medicine and a purpose thereof of the user, and
the nutritional supplement recommendation information comprises a product name, main ingredient, supplementary ingredient, dosage form, a content by raw material, manufacturer, raw material company, brand, product image, selling price, vendor information, and medication guide of a nutritional supplement necessary for the user.

15. The method according to claim 9, wherein the answer data comprises personal preference information comprising at least one of a dosage form, size, odor, medication guide, vegan certification information, gluten-free certification information, and GMO-free certification information of a nutritional supplement.

16. The method according to claim 10, wherein the nutritional supplement comparison information request further comprises product name information of nutritional supplements that the user wants to compare,
wherein the nutritional supplement comparison information comprises ingredient and price comparison information of nutritional supplements corresponding to the product name information.
